(19) United States
(12) Patent Application Publication
(10) Pub. No.: US 2017/0231805 A1
(43) Pub. Date: Aug. 17, 2017

(54) FLUID COLLECTION SYSTEM

(71) Applicant: Gary Lin, Irvine, CA (US)

(72) Inventor: Gary Lin, Irvine, CA (US)

(21) Appl. No.: 15/046,365

(22) Filed: Feb. 17, 2016

(51) Int. Cl. A61F 5/451

(52) U.S. Cl. CPC A61F 5/451; A61F 2005/4402

(57) ABSTRACT

A fluid collection system includes a reservoir element and a receiving element that communicates fluid to the reservoir element. The receiving element includes a first tab, a second tab that can enable the receiving element to stay open on its own during use, and a slot that can receive one of the first and second tabs when the receiving element is in a closed position. The receiving element also includes a first window and a second window, wherein the slot interfaces one of the first and second windows when the receiving element is in a closed position.

### BACKGROUND OF THE INVENTION

The present invention generally relates to fluid collection systems and, more particularly, to apparatus and methods of human urine collection.

Human bodily fluid collection devices collect urine, vomit, or other human bodily fluid from physically challenged persons and immobilized patients. Such fluid collection devices come in different forms and shapes depending on the needs of particular persons.

One prior design includes a urine collection reservoir, a urine collection inlet, a urine collection tube, and an external catheter for male use. The urine collection reservoir is mainly used to store urine up to its capacity and later drained to the sewage system. However, urine may travel through the tube back to the source and cause infection.

Another prior design includes a one-way valve which prevents back flow, and gelling which turns the urine into gelled material and prevents back flow. However, the one-way valve is costly to assemble during production.

Yet another prior design includes a urine-storage bag constructed of a water proof material and snap-fit seal that closes the upper portion of the bag. However, the seal can be difficult to close and there is a risk of contacting the fluid waste.

As can be seen, there is a need for improved apparatus and methods for the collection and disposal of human urine.

### SUMMARY OF THE INVENTION

In one aspect of the present invention, a fluid collection system comprises a reservoir element; a receiving element that communicates fluid to the reservoir element; wherein the receiving element includes: a first tab; a second tab; and a slot that can receive one of the first and second tabs when the receiving element is in a closed position.

In another aspect of the present invention, a fluid collection system comprises a reservoir element; a receiving element that communicates fluid to the reservoir element; wherein the receiving element includes: a first window; a second window; and a slot that interfaces one of the first and second windows when the receiving element is in a closed position.

In yet another aspect of the present invention, a fluid collection system comprises a reservoir element; a receiving element that communicates fluid to the reservoir element; wherein the receiving element includes: a first fold line that extends, along a first direction, from one edge of the receiving element to an opposite second edge of the receiving element; a second fold line that extends along a second direction that is substantially perpendicular to the first direction; a third fold line that extends along the second direction; whereby the receiving element is folded along at least the first fold line when the receiving element is in a closed position.

These and other features, aspects and advantages of the present invention will become better understood with reference to the following drawings, description and claims.

### BRIEF DESCRIPTION OF THE DRAWINGS

FIG. 1 is a perspective view of a fluid collection system, in an open position, according to an embodiment of the present invention;

FIG. 2 is an elevated plan, rear view of the fluid collection system of FIG. 1 in an open position;

FIG. 3 is an elevated plan, rear view of the fluid collection system of FIG. 1 in an open position;

FIG. 4; is a perspective view of the fluid collection system of FIG. 1 in closed position;

FIG. 5 is a perspective view of a receiving element of the fluid collection system of FIG. 1 in a closed position; and

FIG. 6 is a perspective view of the receiving element of the fluid collection system of FIG. 1 in an expanded position for explanatory purposes;

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features. However, any single inventive feature may not address any of the problems discussed above or may address only one of the problems discussed above. Further, one or more of the problems discussed above may not be fully addressed by any of the features described below.

Generally, the present invention provides a fluid collection system that is adaptable for human urine collection. The present invention provides a collection apparatus and method that can be used in different environments—both inside and outside a building. The present invention enables a user to pack and carry the system while utilizing a small amount of weight and size. Similarly, it can be easily used in bed or at a bed side. The present invention can be readily carried in and used in a vehicle. It can also be easily used outdoors, such as for camping.

A receiving element of the present system can be easily opened and held open with one finger while urine is being collected. Once collected, the receiving element can be closed and held in such position by inserting a tab into a slot of the receiving element. A reservoir element that holds the urine can convert the urine to a gel for easy disposal of the entire collection system.

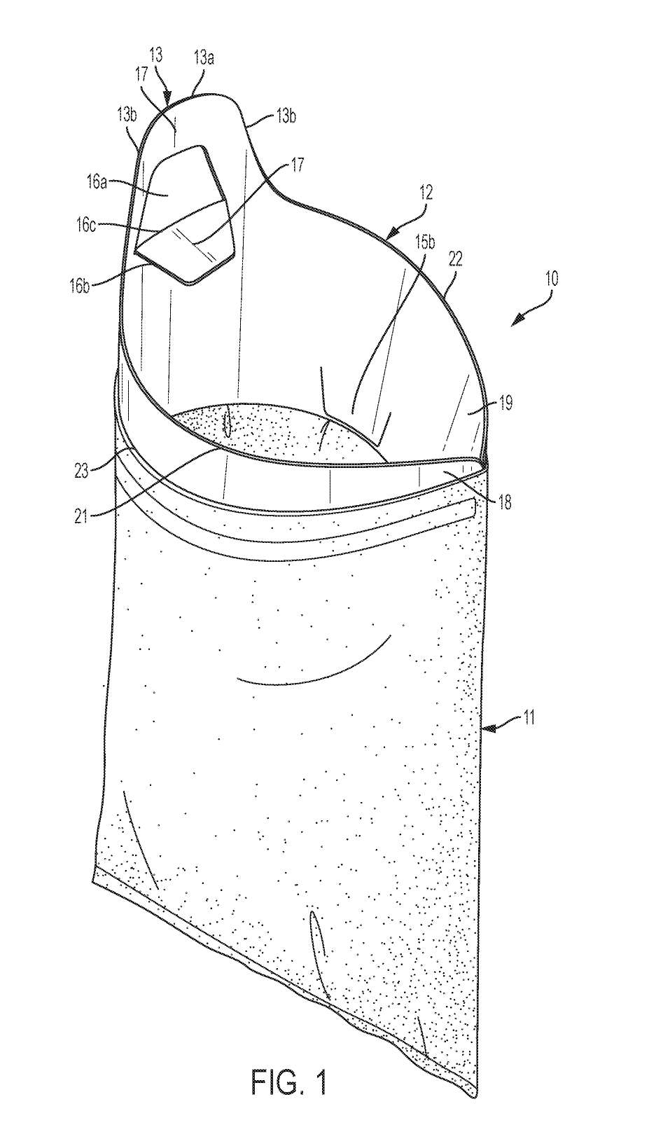

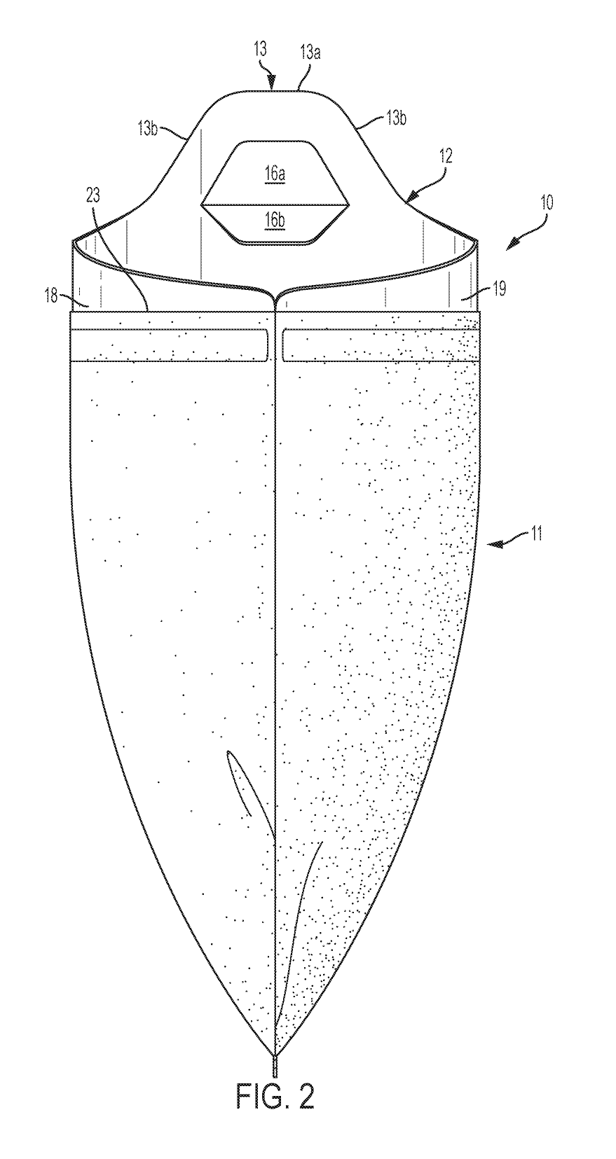

FIG. 1 depicts an embodiment of a fluid collection system 10 in an open position such that fluid, such as human urine, can be collected. The open position of the system 10 is further shown in FIGS. 2 (front view) and 3 (rear view). The system 10 may include a reservoir element 11 and a receiving element 12.

In an embodiment, the reservoir element 11 may be in the form of a bag or pouch and have an opening 23 at one side or edge. The reservoir element 11 can be made of a flexible, lightweight and waterproof material, such as a plastic. In the embodiment shown, the reservoir element 11 may have a generally rectangular configuration, but other configurations can be employed. In various embodiments, the reservoir element 11 can contain a material that, when in contact with urine, can convert the urine to a gel or other solid material. The present invention envisions that the reservoir element 11 can be one as described in U.S. Pat. No. 6,186,990 which is incorporated herein in its entirety.

In an embodiment, the receiving element 12 can interface the reservoir element 11 at the opening 23 so that fluid may communicate from the receiving element 12 and into the reservoir element 11. The receiving element 12, in an open position, can receive the fluid, such as human urine, and enable the user to hold the system 10 before, during, and after fluid collection.

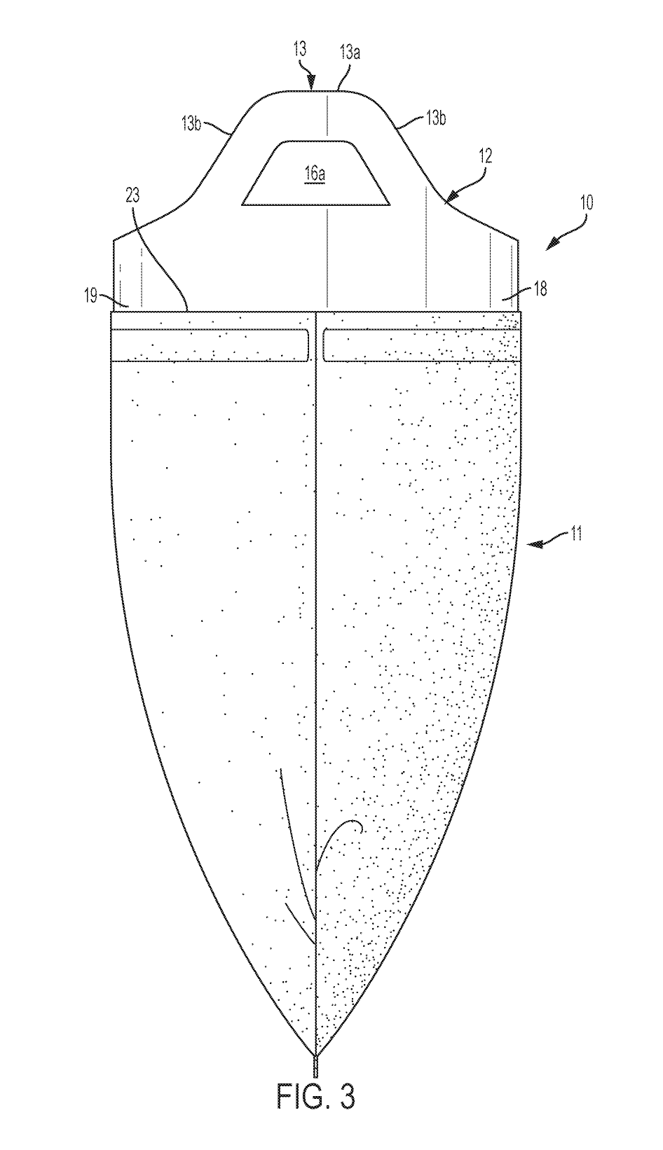

Still referring to FIGS. 1-3, in an embodiment, the receiving element 12 may include a handle portion 13 which can be used by a user, such as one finger of the user, to hold the system 10 before, during and after fluid collection. The handle portion 13 may include a flat segment or edge 13a, a first slanted segment or edge 13b, and a second slanted segment or edge 13b, whereby the first and second slanted segments are on opposed sides of the flat segment 13a.

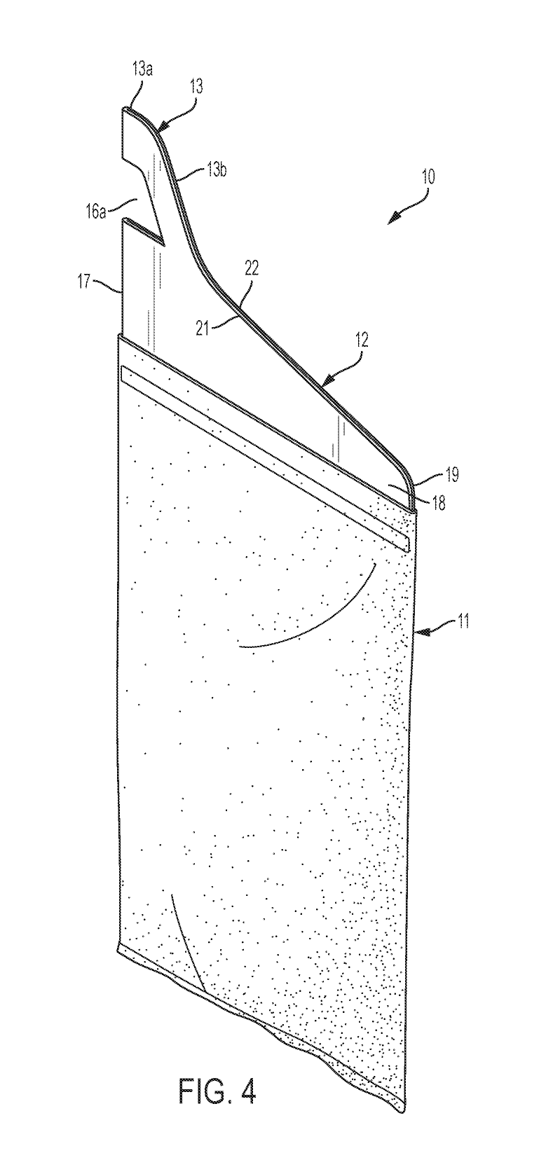

In FIGS. 1-3, and FIGS. 4-5 (closed positions), the handle portion 13 can be disposed between a first shoulder portion or edge 18 and a second shoulder portion or edge 19. A first inclined portion or edge 21 can be disposed between the handle portion 13 and the first shoulder portion 18. A second inclined portion or edge 22 can be disposed between the handle portion 13 and the second shoulder portion 19. A base portion or edge 20 can be on an opposite side of the handle portion 13.

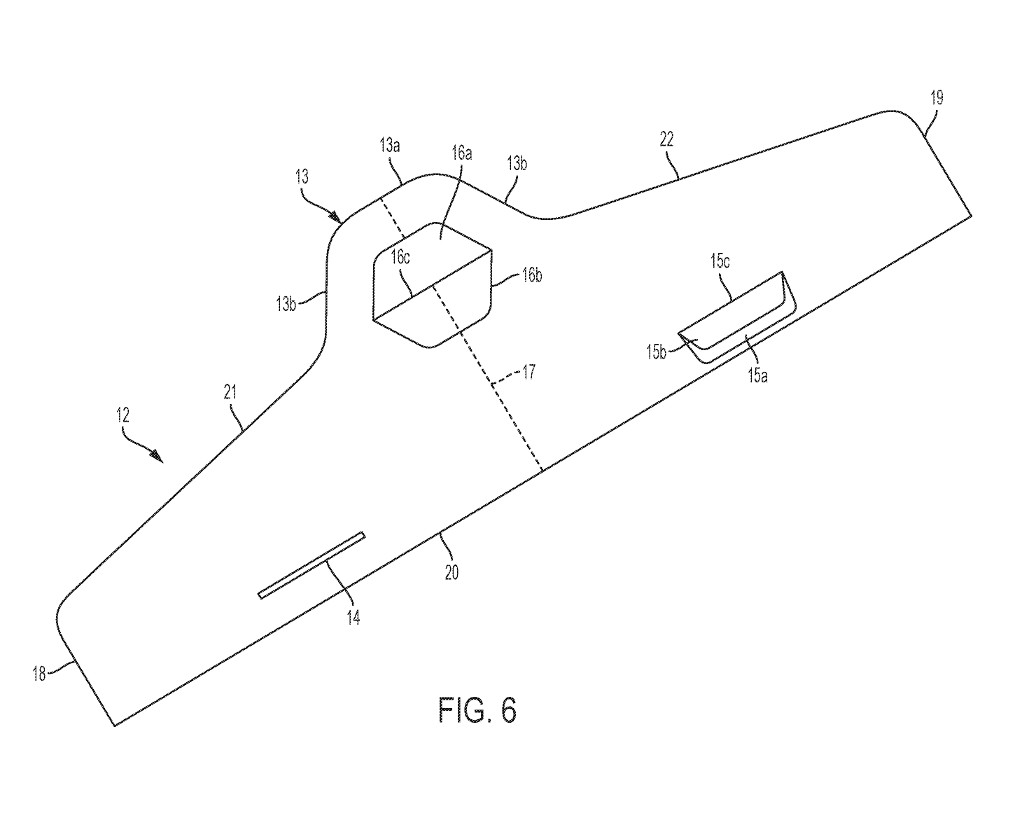

In FIG. 6, in an embodiment, the receiving element 12 may further include a fold or bend line 17 that may extend from the flat edge 13a and to the opposite base edge 20. The fold line 17 may extend and lie along a first direction that can be substantially perpendicular or perpendicular to the base edge 20. In embodiments, the receiving element 12 can be folded or bent along the fold line 17 when the receiving element 12 is moved between open and closed positions. In embodiments, the receiving element 12 can be folded in half.

The receiving element 12 can further include a fold or bend line 15c and a fold or bend line 16c. In an embodiment, a portion of the receiving element 12, such as a first tab 15b, can be folded at and along the first fold line 15c to create a first window 15a. In embodiments, the tab 15b can be folded towards an interior side or area of the system 10, particularly when the receiving element 12 is in an open position.

Similarly, a portion of the receiving element 12, such as a second tab 16b, can be folded at and along the second fold line 16c to create a second window 16a. In embodiments, the tab 16b can be folded towards an interior side or area, or exterior side or area, of the system 10. Moreover, the tab 16b may fold towards and against an interior side or exterior side of the receiving element 12, which serves as an opening force to keep the receiving element 12 in an open position free of any interference, particularly when the receiving element 12 is in an open position. The window 16a can be sized and configured to receive a single finger of a human.

In embodiments, the fold lines 15c and 16c can be substantially parallel or are parallel to one another and lie along a second direction that is substantially perpendicular or is perpendicular to the first direction of the fold line 17. In an embodiment, the fold line 16c intersects the fold line 17. In other embodiments, the fold line 17 bisects the fold line 16c at the midpoint of the latter.

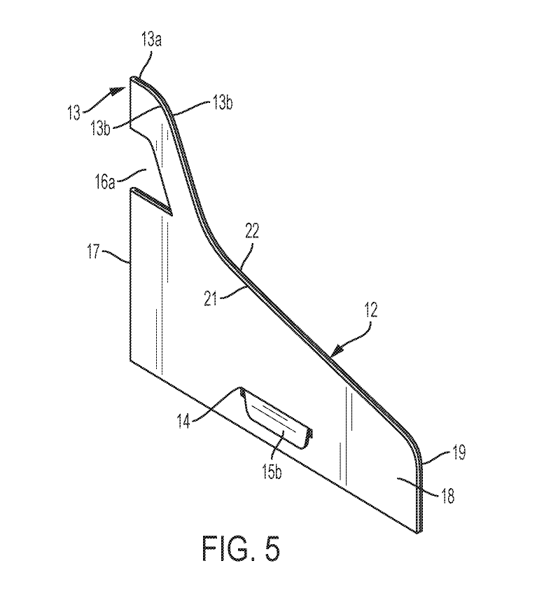

The receiving element 12 may additionally include a slot 14 on a side of the fold line 17 which is opposite a side on which the window 15a can be disposed. Accordingly, when the receiving element 12 is folded along line 17 in a closed position (FIG. 5), the slot 14 interfaces the window 15a and the tab 15b. In embodiments, the slot 14 can receive the tab 15b therein.

In embodiments, the receiving element 12 can be made of a flexible, lightweight, disposable material, such as paper. In certain embodiments, the receiving element 12 may be constructed by stamping paper with the above mentioned fold lines, windows, and slot.

As can be appreciated, when stored, the collection system 10 can be in a closed position and the reservoir element 11 can be folded for greater compactness. For use, the receiving element 12 can be placed in an open position (FIGS. 1-3), the tab 16b can be moved either towards and/or against the interior or exterior side of the receiving element 12, thus enabling the receiving element 12 to stay open on its own. The user can hold the system 10 by one finger in the window 16a. When collection is completed, the receiving element can be moved to a closed position, and the tab 15b may be inserted into the slot 14.

It should be understood, of course, that the foregoing relates to exemplary embodiments of the invention and that modifications may be made without departing from the spirit and scope of the invention as set forth in the following claims.

We claim:

1. A fluid collection system, comprising:
a reservoir element;
a receiving element that communicates fluid to the reservoir element;
wherein the receiving element includes:
a first tab;
a second tab; and
a slot that can receive one of the first and second tabs when the receiving element is in a closed position.

2. The system of claim 1, further comprising a first window at the first tab.

3. The system of claim 1, further comprising a first fold line at the first tab.

4. The system of claim 1, further comprising a second window at the second tab.

5. The system of claim 1, further comprising a second fold line at the second tab.

6. The system of claim 1, further comprising a slot that interfaces one of the first and second tabs when the receiving element is in the closed position.

7. A fluid collection system, comprising:
a reservoir element;
a receiving element that communicates fluid to the reservoir element;
wherein the receiving element includes:
a first window;
a second window; and
a slot that interfaces one of the first and second windows when the receiving element is in a closed position.

8. The system of claim 7, further comprising a first tab at the first window.

9. The system of claim 7, further comprising a first fold line at the first window.

10. The system of claim 7, further comprising a second tab at the second window.

11. The system of claim 7, further comprising a second fold line at the second window.

12. The system of claim 7, wherein the receiving element further comprises a handle portion.

13. The system of claim 12, wherein one of the first and second windows is in the handle portion.

14. The system of claim 7, further comprising a fold line about which the receiving element is folded in half.

15. A fluid collection system, comprising:
a reservoir element;
a receiving element that communicates fluid to the reservoir element;
wherein the receiving element includes:
a first fold line that extends, along a first direction, from one edge of the receiving element to an opposite second edge of the receiving element;
a second fold line that extends along a second direction that is substantially perpendicular to the first direction;
a third fold line that extends along the second direction;
whereby the receiving element is folded along at least the first fold line when the receiving element is in a closed position.

16. The system of claim 15, wherein the reservoir element is in the form of a pouch.

17. The system of claim 15, wherein the receiving element folds in half along the first fold line.

18. The system of claim 15, further comprising a second tab that folds at the second fold line.

19. The system of claim 15, further comprising a third tab that folds at the third fold line.

20. The system of claim 15, wherein the receiving element further comprises a slot that receives a tab.
